(19) United States
(12) Patent Application Publication
(10) Pub. No.: US 2002/0013717 A1
(43) Pub. Date: Jan. 31, 2002

(54) Exercise body monitor with functions to verify individual policy holder and wear of the same, and a business model for a discounted insurance premium for policy holder wearing the same

(76) Inventors: Masahiro Ando, Buffalo Grove, IL (US); Atsushi Hisano, Arlington Heights, IL (US)

Correspondence Address: Thomas J. D'Amico, DICKSTEIN SHAPIRO MORIN & OSHINSKY LLP, 2101 L Street NW, Washington, DC, 20037-1526, US

(21) Appl. No.: 09/749,555

(22) Filed: Dec. 28, 2000

(51) Int. Cl. G06F 17/60; G01C 22/00; A61B 5/00

(52) U.S. Cl. 705/4; 235/105; 600/301

(57) ABSTRACT

An exercise monitor including means to accurately record exercise performed by an insured individual and to generate data needed to calculate an insurance premium. Included are the functions of verifying the person's identity, verifying that he or she is actually wearing the monitor, and allowing a user to self-report his or her encrypted data. Insured clients use inexpensive exercise monitors like pedometers that encourage them to exercise. If a client wears the pedometer and it records results that meet given criteria, the insurance company gives him or her a discount on his or her insurance premium. In this way the insurance company provides an inducement for the insured to adopt a more healthy and active lifestyle. The invention includes a business system to control insurance premiums that enables the company to assume a dominant position with respect to its competitors.

### FIELD OF THE INVENTION

This invention concerns an exercise monitor with the function of verifying a person's identity and the fact that the person is connected to the monitor, and a management system for insurance premiums which employs this monitor. The “function of verifying a person's identity” stated herein refers to determining whether this person is in fact a person previously specified. The “function of verifying the fact that this person is connected to the monitor” refers to determining whether the monitor is actually connected to the body of the specified person. The “management system for insurance premiums” refers to a system by which prospective insurants whose verified exercise test results meet a given standard are offered reduced premiums or other favorable treatment. The aforesaid “given standard” is a standard which makes use of various data to determine whether the person is maintaining or improving his physical and mental health; whether he is preventing illness from occurring; and whether he is promoting his own health.

### BACKGROUND OF THE INVENTION

In recent years premiums for medical insurance have risen steadily, with the employer's contribution becoming larger each year. For management, this uncontrollable increase in expenses poses a vexing problem. Many companies offer their employees health insurance through HMOs (Health Maintenance Organizations) or PPOs (Preferred Provider Organizations), for which the employer assumes a portion of the premium. In this scheme the employee chooses a given medical plan, and from the moment he meets the requirements, he is insured as a member of whichever company group plan he has chosen. His medical expenses are then covered in accordance with specified conditions.

When a person applies to join a health insurance plan, the insurance company will ask him whether he has any pre-existing conditions. By excluding applicants with an extraordinary medical history, the insurance company can avoid having to pay excessive medical expenses.

However, the existing health insurance system ignores efforts by individuals to maintain their own health on a day-to-day basis. The only data that are considered in computing the insurance premium are general criteria such as the prospective insured's age and sex and data concerning that individual's past history.

Yet it is quite clear that the frequency of doctor visits or the frequency of being prescribed medication varies statistically between people who regularly exercise and make an effort to maintain their physical and mental health and people who do not. In other words, an insured person who regularly exercises will incur fewer medical expenses, while a person who does not exercise will incur more. However, once an individual is insured, the effort which he puts into maintaining his health from day to day is not currently reflected in his actual insurance premium. If insured persons were sorted or classed so that their premium reflected how much effort they put into maintaining their own health, not only would the profits of insurance companies rise, but insureds' premiums would be lower. It is also believed that such a scheme would reduce the overall medical expenses of the entire society.

One system which allows individuals' efforts to maintain their health to be reflected in their insurance premiums is the health target control system shown in FIG. 17, which was recently published as Japanese Patent Publication 2000-276525. This invention concerns an insurance product by which an individual's life insurance premium is discounted according to how healthy he is. A person who has signed an insurance contract regularly enters his exercise data via input device 103 at a fitness center designated by the insurance company. Processing unit 109 calculates the person's state of health using various files (not pictured) stored in memory device 102, including file 201, which manages the length of time the person has been studied; file 202, which contains the test results that give the result of the study; file 203, which contains the person's health diagnosis; and file 204, which contains his exercise record. Processing unit 110 calculates the discount rate for that individual's insurance premium, and based on this discount rate calculates his actual premium. It could happen that an insured, in order to receive the premium discount or other special treatment such as various free services, would get a friend to impersonate him and create a spurious exercise record file. However, the health target control system disclosed in Japanese Patent Publication 2000-276525 gives no consideration to preventing another person from assuming the insured's identity and creating false data such as a spurious exercise record file. Since the aforesaid premium discount or other favorable treatment is predicated on the insured's own effort to maintain his health, it is necessary to verify that the person inputting the exercise record is actually the insured and not someone pretending to be the insured. To verify that the insured himself is actually exercising, we must verify both that this is the correct person and that he is exercising. Numerous techniques may be found in the public record to do the single job of verifying the person's identity. For example, in U.S. Pat. No. 4,528,442, applied for by the parent company of the current inventors, the person's answers to a number of questions are compared with previously recorded answers given by the person himself to verify his identity.

However, the current technique to verify identity does not assume the possibility that the person desires that someone impersonate him. For example, pedometers or other sports appliances widely used in fitness centers have the capability of calculating and displaying the amount of exercise a person has completed. An insurance company might trust the amount of exercise recorded by one of these exercise appliances and so discount his insurance premium. The problem here is that in the current system there is no alternative but to accept the self-report from the insured as to how much he has exercised. The insurance company has no means to check the veracity of the self-report. If, for example, an insured records with a pedometer a specified number of steps, and is granted a premium discount, the following problems must be considered.

1) The insurance company has no way to check whether the insured actually wore the pedometer himself or some other person wore it. In other words, it cannot verify the identity of the person exercising. Generally, verifying identity has consisted simply of determining that a person is authorized. For example, it is considered a sufficient proof of identity if the person knows a specific PIN number. For most purposes, it has not been necessary to check whether someone has given his PIN number to a friend who will input it pretending to be him. When the identity of the owner of a bank account is verified, the only question is whether the person is authorized. Since the holder of the account is the only person who stands to gain anything, inputting the correct PIN number is a sufficient identity check. The management system for insurance premiums which is the subject of this invention can verify that the person who reports the amount of exercise is authorized. But if the insurance company cannot verify sufficiently that this person is not someone pretending to be the insured, this system cannot fulfill the requirement of verifying that the insured himself is making an effort to maintain his health. In other words, the existing verification technique does not fulfill the two necessary conditions, i.e., it must verify both that this is an authorized person and that this is not someone other than the insured.

2) If swung by hand, a pedometer will count the movements as steps even though the person is not walking or jogging. There is no way to verify that the pedometer is actually attached to the person's body, or whether it is attached to anything. Let us assume that an insured is paying a monthly premium of $700, which comes to an annual outlay of $8,400. If he does a specified amount of exercise, he will receive a discount of 10%, or $840 per year. It is not inconceivable that the insured will use the pedometer improperly and actuate it simply to log the required number of steps. He might, for example, attach the pedometer for a calculated period of time to something that revolves mechanically. Obviously, this sort of spurious exercise record should not be counted as an effort on the part of the insured to maintain his health. Yet the existing technique entails no actual means to verify that the pedometer is attached to the insured's body.

### SUMMARY OF THE INVENTION

As is discussed above, since regular exercise is linked to maintaining or improving physical and mental health, preventing illness and promoting a healthy lifestyle, some insurance companies have a policy of granting benefits such as discounts to insureds who exercise. This invention is a system designed to be used by such companies. More specifically, the invention concerns an exercise monitor which is a means to accurately record exercise results consisting of the amount of exercise the insured himself has performed.

The exercise data to be recorded include at least the type of exercise, intensity level and length of time. These data will be expressed in terms of type and amount of exercise. Some examples of types of exercise are jumping, walking, jogging and jumping rope. Some examples of intensity level are height of jumping, speed of walking or jogging and r.p.m. of the rope for jumping rope. Length of time is simply how long one exercises. It can be expressed by logging the starting and finishing times. The amount of exercise can be expressed using the intensity and length of time. For walking, it would be proportional to distance walked or number of steps taken.

The “exercise results” are the record of exercise performed. For example, the results might be all the daily exercise data recorded in the last thirty days.

To verify reliably that the exercise record represents exercise performed by the insured himself, it is necessary to verify at least that the person monitored is in fact the insured. In fact, as can be seen in the following chart, to assure ourselves that the exercise record is accurate we need to verify two points-that this person is actually the insured, and that the exercise monitor is actually attached to his body.

| Verifying | | |
|---|---|---|
| that the | | |
| person is | | |
| actually | Verifying that the exercise monitor is actually | |
| the insured | attached to the person | |
| Means | | |
| Use of | Have a third | Data will not be generated if ex- |
| identity | party verify | ercise monitor is not attached to |
| checking | that the exer- | correct person. Verify that data |
| techniques | cise monitor is | are being generated. |
| | attached to the | |
| | insured. | 1. Verify that there are data. |
| 1, Answers | | Since data will not be detected |
| to ques- | 1. Rely on staff | unless monitor is attached to |
| tions | at fitness cen- | correct person, display on moni- |
| which only | ter to verify | tor represents correct use. (Ex- |
| insured | that the person | ample: Attendant may feel moni- |
| can an- | exercising is | tor. If vibrating, it is re- |
| swer; in- | wearing the ex- | cording.) |
| put of PIN | ercise monitor. | |
| number. | | 2. Verify detection of specific |
| | | signal which is only emitted when |
| | | person is exercising while wear- |
| 2. Input | 2. Rely on | ing monitor. (Example: signal |
| of physi- | trainer to ver- | emitted when person is walking) |
| cal data | ify that person | |
| which can | is wearing moni- | 3. Verify physiological data with |
| identify | tor. | characteristics which cannot be |
| an indi- | | input into monitor unless it is |
| vidual, | | worn while the person is exercis- |
| such as | | ing. (Example: a pulse wave |
| finger- | | with a component highly corre- |
| print or | | lated with the rhythm of sounds |
| voice- | | indicating that the person is ex- |
| print. | | ercising.) |
| | | 4. Verify acceleration data with |
| | | characteristics which cannot be |
| | | input into monitor unless it is |
| | | worn while the person is exercis- |
| | | ing. (Example: capturing an ac- |
| | | celeration signal with a compo- |
| | | nent highly correlated with the |
| | | rhythm of sounds indicating that |
| | | the person is exercising.) |

| How Means Should be Combined | |
|---|---|
| 1. | The identity of the person and the fact that the |
| | monitor is attached to his body should be verified |
| | at nearly the same time. (This will prevent the in- |
| | sured from (1) transferring his monitor to a friend |
| | after his identity is confirmed and having the |
| | friend submits to the check of whether the monitor |
| | is attached, or (2) having his friend submit to the |
| | check of whether the monitor is attached and then |
| | transferring the monitor to himself for the identity |
| | check.) |
| 2. | The monitor should be watched from the time the per- |
| | son's identity is checked until the check that he is |
| | wearing the monitor is completed to make sure that |
| | he does not remove it. |
| 3. | The monitor should be watched from the time the per- |
| | son is checked to make sure he is wearing it until |
| | his identity check has been completed to make sure |
| | that he does not remove it. |

Technology needed to combine means
Techniques to detect removal of monitor:
Continuously detect cardiac pulse. If pulse is not de-
tected for a given period of time, conclude that moni-
tor has been removed. Monitor will store last time
pulse was detected and calculate difference between
this time and the next time pulse is detected. If
elapsed time exceeds a given value, conclude that moni-
tor has been removed.

The first objective of this invention is to provide an exercise monitor which could generate data needed to calculate an insurance premium which would be beneficial and fair to both the insurance company and the insured. To enable the exercise monitor to record accurately only exercise performed by the insured himself, this invention has been given the two basic functions described in the above chart, namely the function of verifying the person's identity and the function of verifying that he is actually wearing the monitor; and it has been given the additional function which allows the user to self-report his encrypted data.

Insurance companies might provide their clients with inexpensive exercise monitors like pedometers and encourage them to exercise. If a client wore the pedometer and it recorded results that met given criteria, the insurance company could conclude that he had made an effort to maintain his health and would give him a discount on his insurance premium. In this way the insurance company could provide an inducement for the insured to adopt a more healthy and active lifestyle.

The second objective of this invention is to provide a business system to control insurance premiums. If it uses an exercise monitor as described above to accurately record an insured's efforts to maintain his own health, the insurance company will be able to reduce his premium. This advantage will enable the company to assume a dominant position with respect to its competitors. To achieve the two objectives stated above, this invention is given the following two preferred embodiments.

The first preferred embodiment of this invention consists of an exercise monitor and a management system for insurance premiums in which the identity of the person and the fact that he is wearing the exercise monitor are verified by one subsystem and the exercise record data are captured and evaluated by another subsystem, both of which are connected to the monitor via a communication circuit. The first step in the process is for the insurance company to contact by telephone or other means of communication the person who has purchased a health or life insurance policy and verify his identity. Shortly after or shortly before this contact, exercise record data recorded by an exercise monitor (for example, a pedometer) worn by the insured are taken and analyzed to verify that he has actually worn the monitor. If the exercise indicated on the monitor at the time the person's identity is verified (for example, light jumping or walking some number of steps) can be verified by subsequent processing to be exercise performed by the person at that time, we may conclude that the person was wearing the monitor. The exercise record data from the exercise monitor are analyzed to determine whether the person has performed sufficient exercise to meet the requirements to have his premium discounted. If the recorded exercise does meet the requirements, the identity check and the monitor check are judged to be complete and the appropriate steps are taken to discount the premium. The insured may also be given guidance by a trainer regarding his exercise routine based on the exercise record data captured by his monitor.

The second preferred embodiment of this invention is an exercise monitor which has a built-in program to check the person's identity, check whether he is actually wearing the monitor, and evaluate his exercise; and a management system for insurance premiums employing that monitor. In the second embodiment, the person's identity and the fact that he is wearing the monitor are verified according to the following steps.

1) The exercise monitor keeps track of the person's exercise and stores the data in a non-volatile memory. (Examples: Number of steps, list of dates and times steps were counted)

2) The exercise monitor detects when the person is resting after exercising. (Example: When there is a period of time that the acceleration is below a threshold value which continues until the next time the threshold is reached.)

3) When the exercise monitor has determined that the person is resting after exercising, it will issue a request that the person verify his identity. (This may be done at every rest period or without notice.) He will then be required to input data to verify his identity. Data that he might be asked to input include PIN number, date of birth, or answers to simple questions, such as favorite color, blood type, animal in Chinese zodiac, astrological sign, or place of birth. By having the monitor verify the person's identity immediately after verifying that he has just finished exercising, we can detect that the person wearing the monitor is really the insured. The identity check is not requested while the person is exercising so that his exercise will not be interrupted. A device may also be used which can verify identity using physiological data. In this case, since the check can be done without disturbing the person while he is exercising, the identity check need not be limited to the period immediately after the person exercises. To increase the accuracy of the check to see if the person is wearing the monitor, the monitor can request that the person perform a simple action (e.g., jump or walk a few steps). If the action is detected immediately after the request is made, we can conclude that the person is wearing the monitor. If a number of different questions are stored in the monitor and selected randomly, and the check of whether the person is wearing the monitor is performed shortly before or after the time his identity is checked, the insurance company can increase the probability that the person wearing the monitor is actually the insured and not a friend helping him out. At the same time, as was discussed above, we can verify that he is actually wearing the monitor.

4) The exercise monitor compares the exercise record data which it has recorded with a given criterion. If the data fulfill this requirement, it uses the monitor owner's ID to generate a PIN number and display it on the screen. Along with the PIN number, the monitor displays the insurance company's toll-free phone number. It may also explain to the insured that if he calls the toll-free number and reports this PIN number, his premium will be discounted. The information could also be sent via the internet using a data input section on the insurance company's home page. This would allow the insured to send the insurance company accurate and reliable exercise data.

The second embodiment of the exercise monitor does not need to have a transmitting function built into the monitor, and it does not require the user to exercise in a place with a phone or internet connection. The processing is done off line, so the user has greater freedom to exercise when and where he chooses than he would with the first embodiment. This design also reduces the cost and size of the monitor.

### BRIEF DESCRIPTION OF THE DRAWINGS

FIG. 1 illustrates an overview of the exercise monitor according to the first preferred embodiment of this invention.

FIG. 2 illustrates an overview of the exercise monitor according to the second preferred embodiment of this invention.

The FIG. 3 illustrates the hardware configuration of the exercise monitor according to the second preferred embodiment of this invention.

FIG. 4 illustrates the partial software flowchart of main routine of the exercise monitor according to this invention.

FIG. 5 illustrates the partial software flowchart of main routine of the exercise monitor according to this invention.

FIG. 6 illustrates the partial software flowchart of main routine of the exercise monitor according to this invention.

FIG. 7 illustrates the partial subroutine SUB 200, the processing to verify that the monitor is being worn, according to this invention.

FIG. 8 illustrates the partial subroutine SUB 200, the processing to verify that the monitor is being worn, according to this invention.

FIG. 9 illustrates the partial subroutine SUB 200, the processing to verify that the monitor is being worn, according to this invention.

FIG. 10 illustrates the partial subroutine SUB 200, the processing to verify that the monitor is being worn, according to this invention.

FIG. 11 illustrates the partial subroutine SUB 200, the processing to verify that the monitor is being worn, according to this invention.

FIG. 12 illustrates the partial subroutine SUB 300, the processing to verify the user's identity, according to this invention.

FIG. 13 illustrates the partial subroutine SUB 300, the processing to verify the user's identity, according to this invention.

FIG. 14 illustrates subroutine SUB 400, which concerns recording the type, amount and time of exercise.

FIG. 15 illustrate insurance premium management system 500, which is related to the third preferred embodiment of this invention.

FIG. 16 illustrates insurance premium management system 600, which is related to the fourth preferred embodiment of this invention.

FIG. 17 illustrates the health target control system, which is a prior art, and was recently published as Japanese Patent Publication 2000-276525.

### DETAILED DESCRIPTION OF THE INVENTION

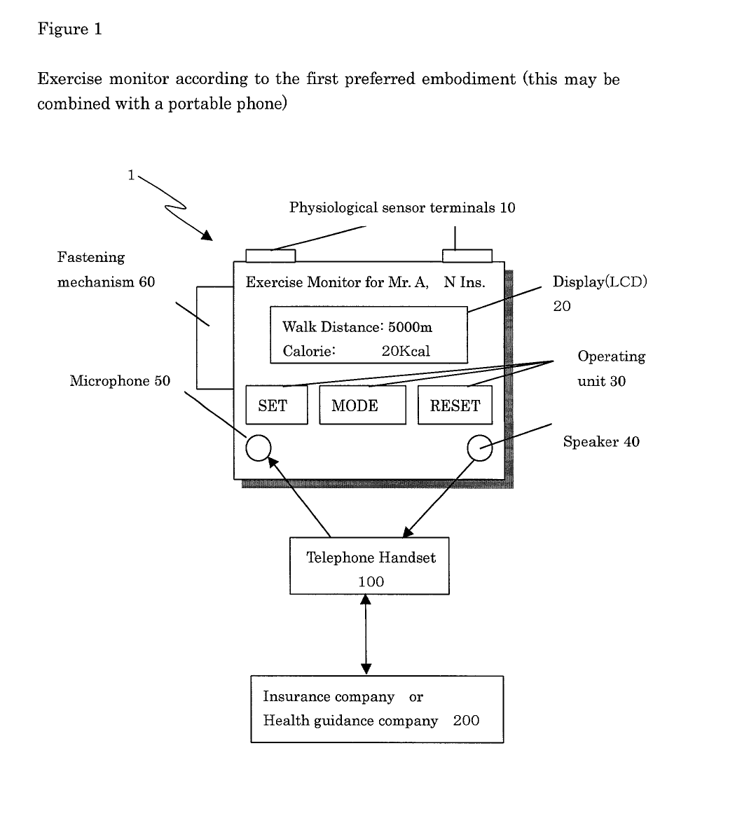

FIG. 1 shows the exterior of exercise monitor 1, the first preferred embodiment of this invention.

This type of exercise monitor is fastened by fastening mechanism 60, a belt or the like, to the upper or lower portion of the person's thigh. It records what type of exercise he is doing and measures its amount and duration, storing all the data along with the date and time. The exercise monitor may be combined with a portable phone so that the data detected by the monitor may be sent to a remote location or data such as commands or questions may be sent from a remote location and displayed on the monitor. Data which are measured and recorded could also be processed and displayed on display 20. Sensor terminals 10, which measure physiological data, and/or an acceleration sensor (not pictured), are built into the exercise monitor. The monitor also contains a program which uses the sensor data to determine when the person is exercising and calculate the amount of exercise (e.g., in number of steps) he is getting, which is the reason for the monitor.

The exercise monitor has the capability of verifying that the person who is wearing it is actually the insured and not someone impersonating him (the identity-checking function). This prevents the insured from illegally procuring favorable treatment such as a rate discount by having someone else wear the monitor while exercising. The insurance company (or the health guidance company) calls the person on telephone 100 without notice and asks him questions about himself which must have specific answers. Questions that he might be asked in an identity check include PIN number or date of birth, or questions that the insured would be able to answer easily without thinking, such as favorite color, blood type, animal in Chinese zodiac, astrological sign, or place of birth.

If the insured's identity can be verified from his answers to these questions, the monitor checks whether he is actually wearing the monitor or has rigged it in some way to clock spurious exercise (function of verifying that the person is wearing the monitor). For example, a representative of the insurance company (or health guidance company) contacts the insured by phone and asks him to jump a few times, and the insured does so. If the exercise monitor is part of a cell phone, the entire exercise routine can be directed and monitored in real time.

Next, the insurance company's (or health guidance company's) computer takes in the data collected by the exercise monitor and transmitted by phone. When the exercise data representing the jumping which has just occurred are output and the insured's ID has been detected, those data are processed as representing exercise performed while the insured is wearing the monitor. If the exercise monitor is not connected by a circuit to the computer at the insurance company (or health guidance company), that computer will be unable to evaluate the exercise data recorded by the exercise monitor. In this case, the exercise monitor can compare the exercise data it has recorded with an appropriate standard. If the data meet the standard, encrypted data generated by using the ID of the person using the exercise monitor can be displayed on screen 20. Since the data are encrypted, it is not possible for the person to report fraudulent data to the insurance company (self-reporting function of encrypted data). When this exercise record is transmitted to the insurance company, if the exercise recorded is sufficient to meet a given standard, the insured receives the service he has been promised, such as having his premium discounted.

In this device, in addition to the features described above, operating unit 30 executes the input shown in the next flow chart. Also, the device and the person can exchange data via speaker 40 and microphone 50, or either can exchange data with the insurance company by phone. Alternatively, the insured can use the internet to access a data reception page on a given home page set up by the insurance company and report the aforesaid encrypted data.

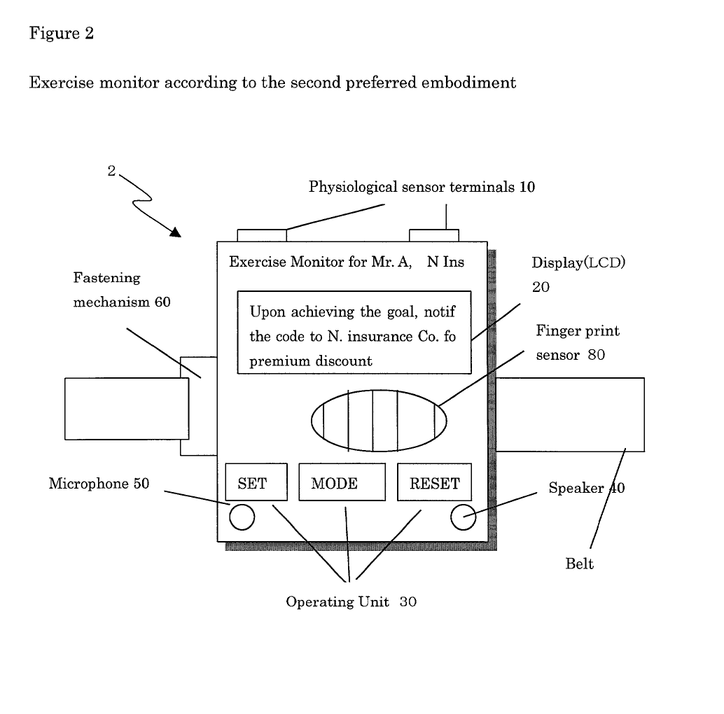

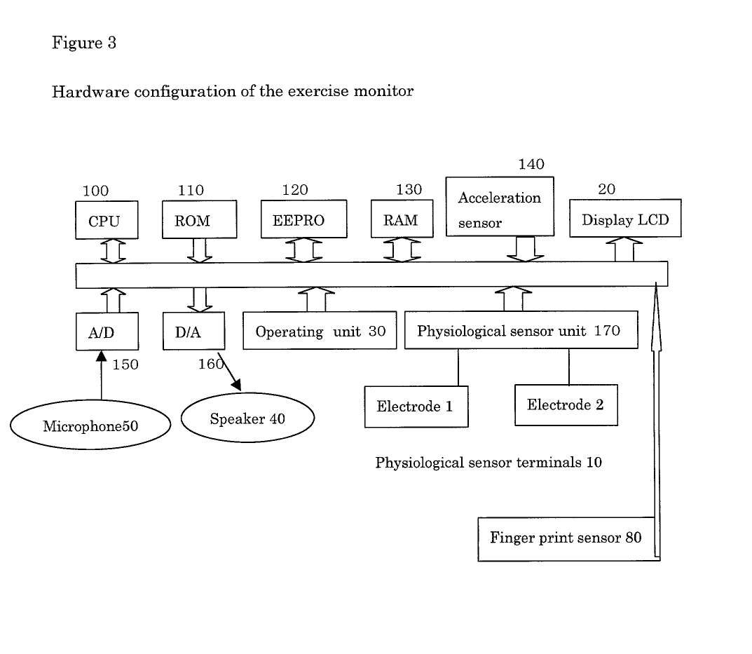

FIG. 2 shows the external appearance of exercise monitor 2, the second preferred embodiment of this invention.

In the second embodiment, in contrast to the first, the person's identity and the fact that he is wearing the monitor are not confirmed by calling the insurance company on the telephone, but by exercise monitor 2 itself. First the person wearing the device is monitored and the data are recorded. Then, immediately after the monitor detects that exercise has ceased, an internal program runs which instructs the person to perform some light exercise (for example, a given number of jumps) to check whether the monitor is being worn. The exercise data for the given number of jumps are collected by sensor terminal 10 for physiological data or by an acceleration sensor. The monitor determines whether a characteristic signal pattern has been detected which is generated during exercise due to the skeletal or muscle structure of the human body, and from this result it determines whether the exercise monitor is being worn by a human being. In other words, the monitor can check whether someone is wearing it by comparing the current data with previously recorded reference data (function to check that monitor is being worn).

Next, an internal program which receives data input for the identity check (for example, fingerprint input or voice input) runs to verify the insured's identity (identity check function). The items covered by the identity check are the same as in the first embodiment; however, fingerprint sensor 80 may be used and the results compared with reference data stored in the device or specific buttons on operating unit 30 may be used to input a PIN number. An example of a small fingerprint sensor which may be used is Sony's model CXA3271GE. Alternatively, microphone 50 may be used to input the person's voice, and the monitor can then determine whether the voiceprints match. In any case, unlike in the first embodiment, the monitor itself checks whether the person is wearing the monitor and who that person is, and both checks are performed offline.

When the monitor has completed the checks of whether the person is wearing the monitor and who the person is, and the exercise record data which have been recorded meet the given standard, just as in the first embodiment encrypted data generated using the insured's ID are displayed on screen 20. The insured uses the phone or a data reception page on a given home page set up by the insurance company to report the aforesaid encrypted data (function of self-reporting encrypted data). Encryption of the data prevents fraudulent reporting just as in the first embodiment.

Hardware

We shall next explain the hardware configuration of exercise monitor 1 or 2 of this invention, with reference to FIG. 3. The main internal components are CPU 100, ROM 110, EPROM 120, RAM 130, acceleration sensor 140, A/D converter 150, D/A converter 160 and unit 170 to measure physiological data. The main external components are sensor terminal 10 to measure physiological data, which was mentioned above; display unit 20; operating unit 30; speaker 40; microphone 50; and fingerprint sensor 80, which is used in the second embodiment. All components are connected by bus line 105.

All programs related to the exercise monitor are stored in ROM 110. The ROM also contains basic wave form patterns for different types of exercise, threshold values for determinations and control parameters. EEPROM 120 contains the ID number of the exercise monitor; the name and PIN number of the monitor's owner; the questions used to verify the user's identity and the correct answers to them; and physiological data needed to verify the user's identity, such as their fingerprint pattern or voiceprint pattern. These data are entered into the EEPROM before the monitor is given to the insured based on data supplied directly by that person. RAM 130 is a memory area used as a work area for required software processing. Display unit 20 consists of an LCD which displays messages to the user who is operating the device. This display is often generated synchronously with the sound from speaker 40. Unit 170 to measure physiological data could, for example, be used to obtain the waveform of an ECG or the cardiac rhythm. Operating unit 30 consists of a set of buttons which are used to switch modes or enter answers to questions, input a PIN number, and so on. Fingerprint sensor 80 is used to input the user's fingerprint into the monitor.

Software Flow Chart

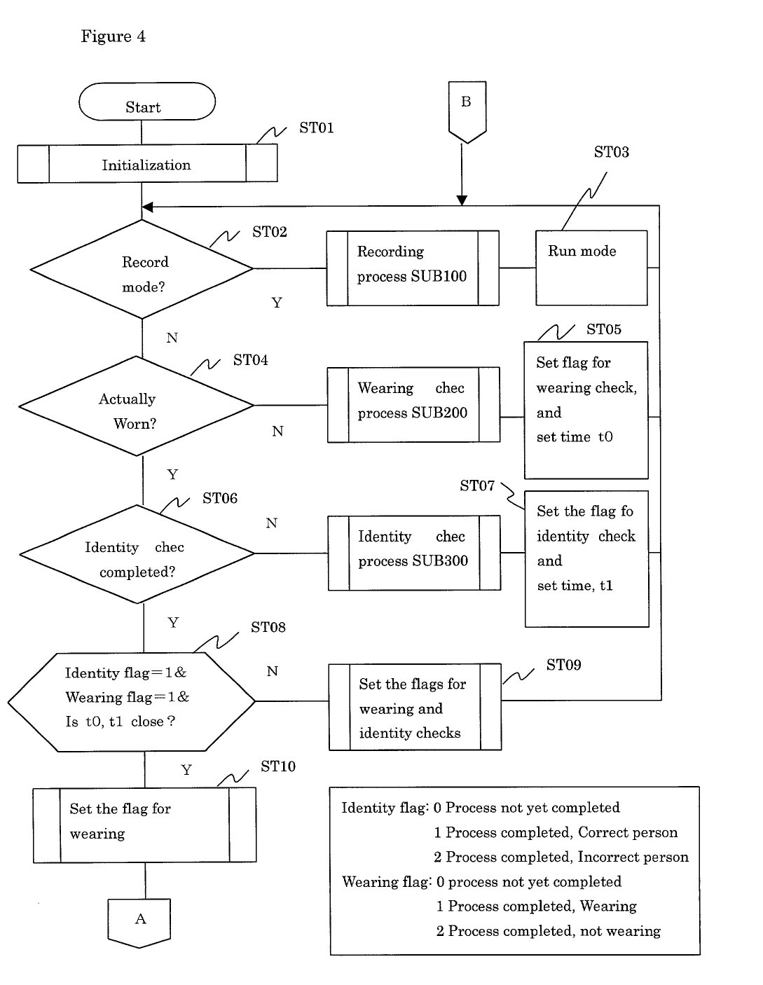

We shall next explain a flow chart for the software operations executed in an exercise monitor related to this invention, with reference to FIGS. 4 through 14. Since many aspects of exercise monitors 1 and 2 in the aforesaid first and second embodiments overlap, we shall discuss only the second embodiment in this section.

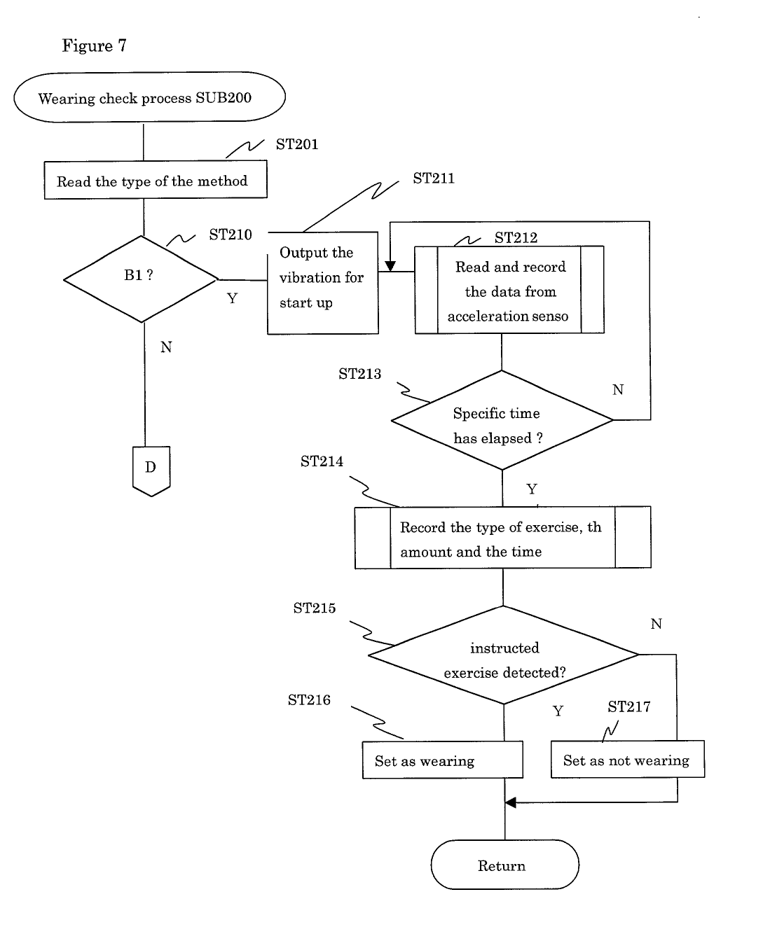

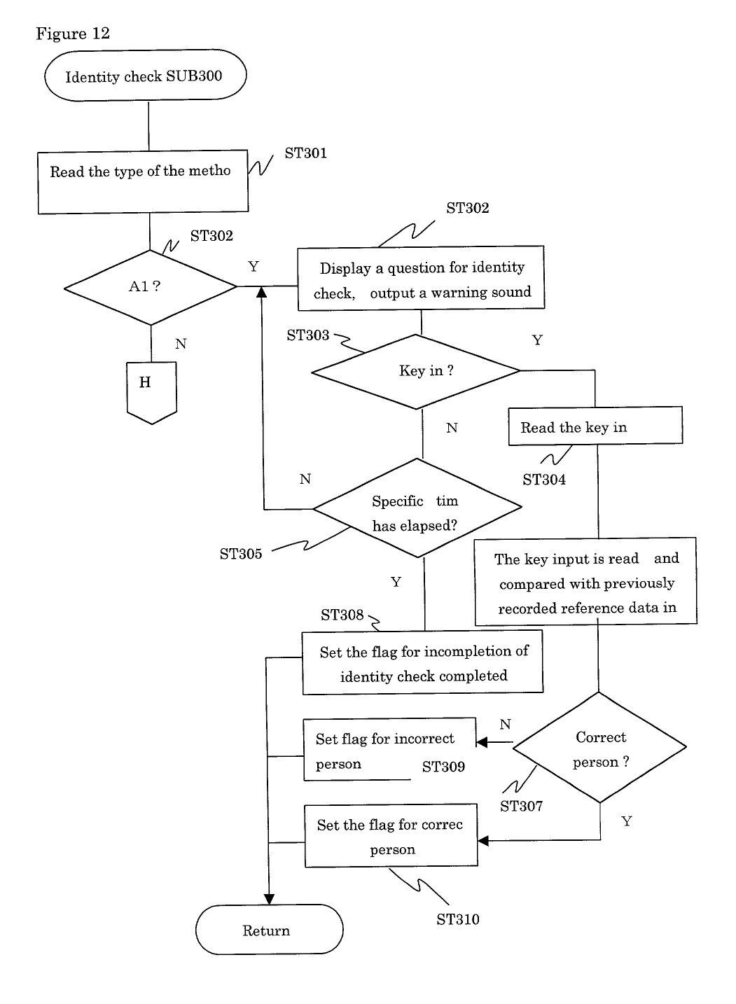

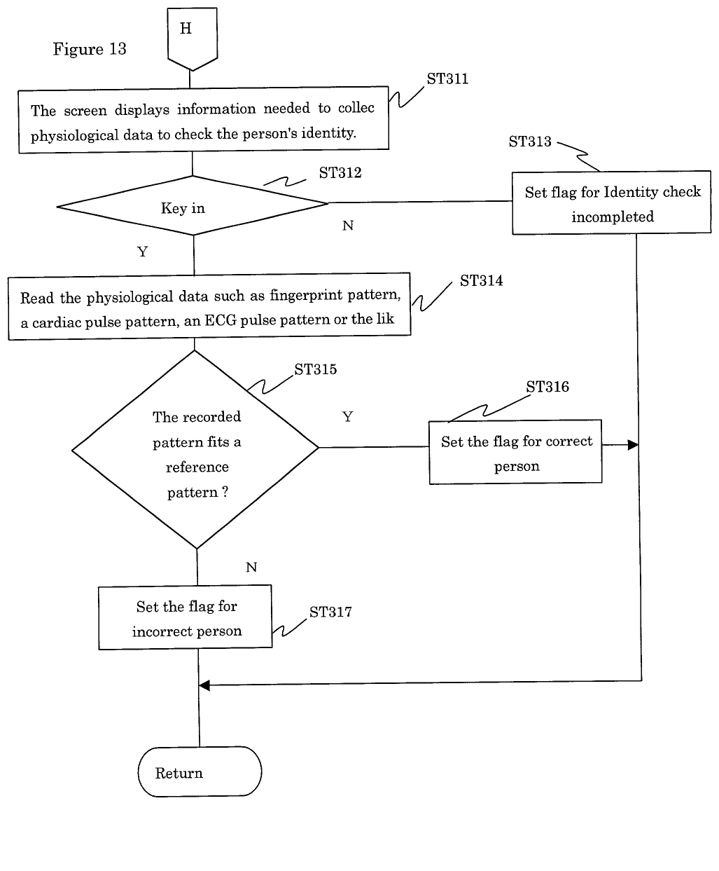

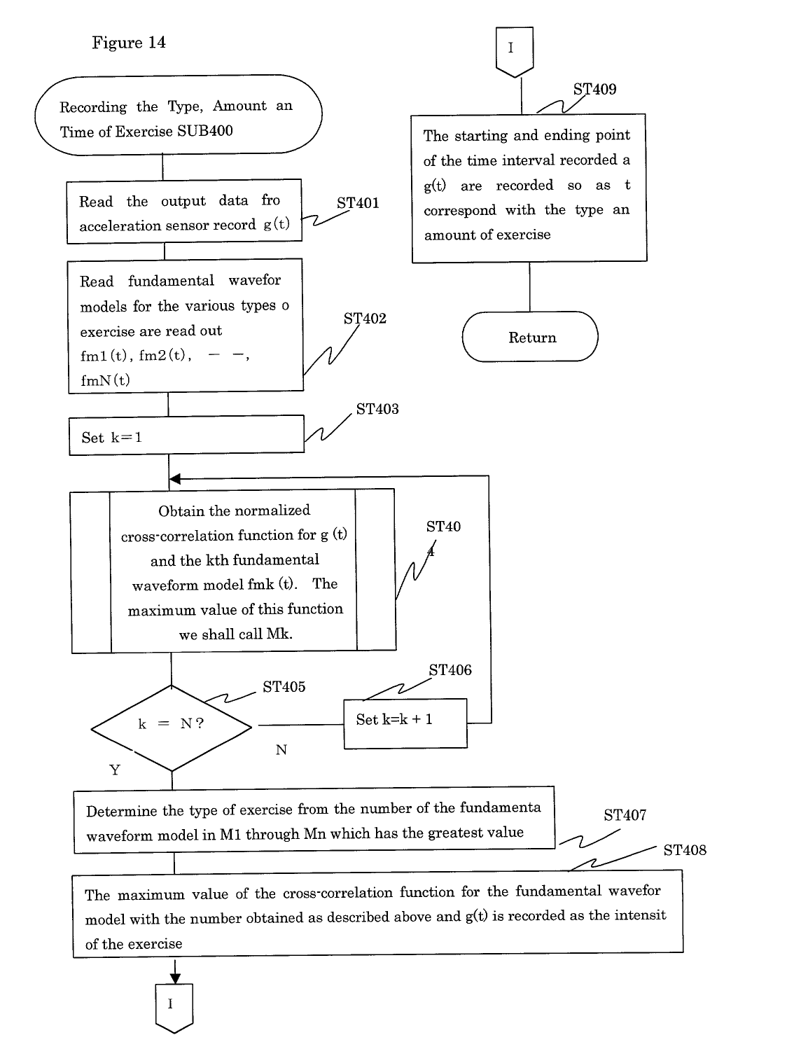

FIGS. 4 through 6 outline the main routine. FIGS. 7 through 11 illustrate subroutine SUB 200, the processing to verify that the monitor is being worn. FIGS. 12 and 13 illustrate subroutine SUB 300, the processing to verify the user's identity. FIG. 14 illustrates subroutine SUB 400, which concerns recording the type, amount and time of exercise.

Verifying that the Monitor is Being Worn and Verifying Identity

First, as can be seen in FIG. 4, when exercise monitor 1 or 2 is fastened to the specified part of the insured's body, a series of initialization processes is executed in Step 01. In Step 02, the monitor checks whether the “mode” button on operating unit 30 is set to “record mode.” Record mode is the mode in which data are recorded to verify the user's identity and evaluate his exercise. If the monitor is in record mode, specific recording processing is executed in subroutine SUB 100. When this is completed, the monitor is set to “run mode” in Step 03. Again the monitor executes Step 02 and finds that it is now in run mode rather than record mode.

We proceed to Step 04, where the monitor checks whether the processing has been completed to verify that it is actually attached to someone's body (processing to verify that the monitor is being worn). This processing confirms that the exercise monitor is really attached to the person's body and is not being operated by some improper means. If the “monitor is being worn” flag is “0”, the verification processing is not yet completed; if it is “1”, the processing has been completed. If the flag goes to “2”, this indicates that the processing is completed and the monitor is not being worn. Normally, when record mode has just been completed, the monitor will determine that the check of whether the person is wearing it has not yet been completed. It will then execute subroutine SUB 200, to be discussed shortly, to check whether the monitor is actually attached to a person. In Step 05, the “monitor being worn” flag is set according to the result of the check, and the time of the check to is set. We again proceed through Steps 02 and 04, and check in Step 06 whether the identity verification has been completed. If the check of whether the monitor is being worn is still not completed, we go again to SUB 200.

If the check of whether the monitor is being worn has been completed in Step 04, we proceed to Step 06 and check whether the person wearing the monitor is actually the insured and not someone not authorized to wear the monitor (processing to verify identity). This processing to verify the insured's identity is done to certify that this is indeed the insured and not someone to whom he has improperly lent his exercise monitor. If in Step 06 it is determined that the person's identity has not yet been verified, we go to subroutine SUB 300, which will be discussed shortly, and the specified processing is executed to verify the person's identity. In Step 07, the identity check flag is set according to the result of this processing, and the time t₁ of the check is set. We then return to Step 02. An identity check flag of “0” indicates that the verification processing is not yet completed; “1” indicates that the processing has been completed and the insured's identity has been confirmed; and “2” indicates that the processing has been completed and someone other than the insured is wearing the monitor.

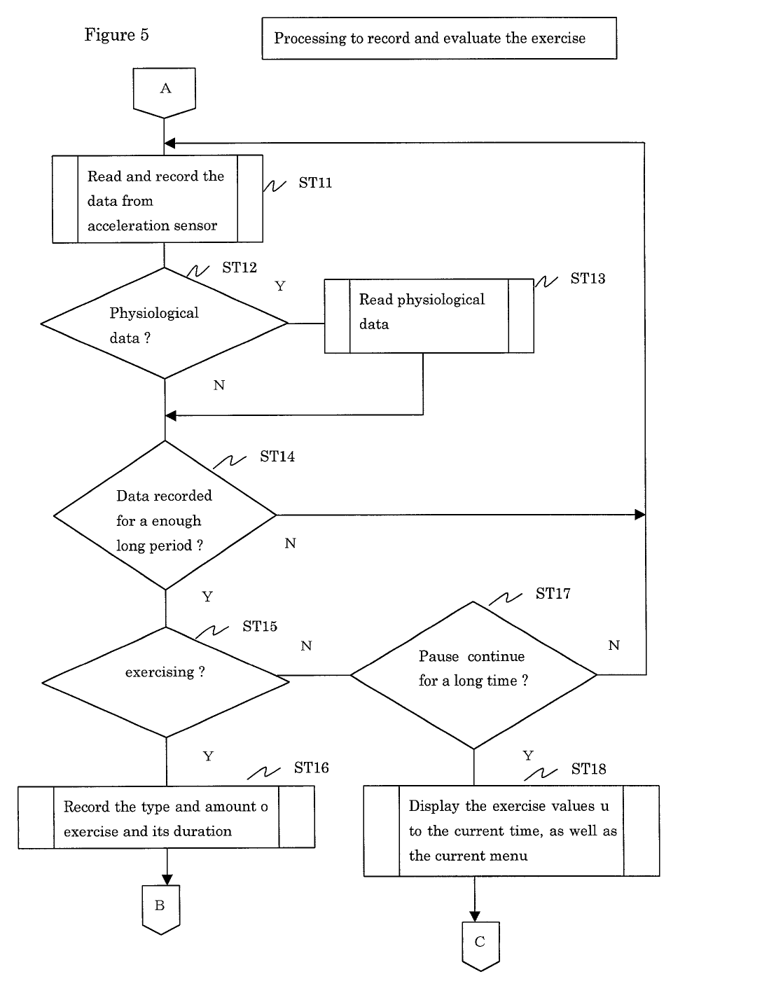

If in Step 06 it is determined that the identity check processing has been completed, we go to Step 08, where it is determined whether times t₀ and t₁, the times when the identity check was completed (when the flag went to “1”) and when the check of whether the monitor was being worn was completed (when the flag went to “1”) are sufficiently close to each other. If they are, the monitor concludes that the monitor was not removed after the identity check. In Step 10, the “worn by insured” flag is set, and since the processing to verify that the monitor is being worn and that the correct person is wearing it has been completed, we proceed to the processing to record and evaluate the exercise shown in FIG. 5. If times t₀ and t₁ are not sufficiently close to each other in Step 08, the monitor concludes that it is being used improperly. the flags are reset in Step 09, and we return to Step 02.

The Processing to Record and Evaluate the Exercise

In the processing to record and evaluate the exercise which is shown in FIG. 5, the amount of exercise is recorded for the person for whom two conditions have been verified: the condition that he is wearing the monitor on his body, which was verified in the aforesaid SUB 200, and the condition that he is actually the insured, which was verified in SUB 300.

In Step 11, data are recorded from an acceleration sensor which is built into the monitor. By “acceleration sensor,” we mean merely a sensor which detects acceleration associated with the movement of the body. For example, if we were to choose a pedometer, the pendulum-type sensor used in the prior art would be suitable. If the person was planning to use the various machines in a health club, a sensor or counter to detect the characteristic movement of various parts of his body could be used. Since various kinds of sensors and counters are already available, we shall not discuss them in detail at this point. Since the point of the invention is to measure an amount of exercise effective in maintaining the health of an insured person so that his insurance company can confer benefits on him, the sensor must be a measurement device capable of measuring such an amount of exercise. The invention is not necessarily limited to a single measurement device; a number of such devices could as well be used.

In Steps 12 and 13, read-in mode is used to read in the physiological data and store them. By “physiological data” are meant such data as heart rate and blood pressure. An example of a sensor for physiological data is the sensor terminal 10 shown in FIG. 2; however, the physiological data are not limited to this type only, but include any data detected by any of various physiological sensors, including a microphone within a cuff and a laser sensor stuck to the skin. In Step 14, the monitor checks whether the data have been recorded for a period which is long enough to evaluate them. If the time period is too short, we return to Step 11. If it is adequate, we go to Step 15, where, based on the data recorded in Steps 11 and 13, the monitor determines whether the user is now exercising or has temporarily stopped. If it determines that the person is exercising, it records in Step 16 the type and amount of exercise and its duration. We then return to Step 02 and continuously repeat the check to verify that the monitor is being worn, the identity check, and the steps to record the exercise until the monitor determines in Step 15 that the person has stopped exercising. If it determines in Step 15 that the person has stopped, but the pause is within a given duration, the monitor will conclude that the person has taken a short rest, and will return to Step 11 and resume recording the exercise. In Step 17, if the pause continues for a long time, the monitor will conclude that the person has finished exercising and is now resting. In Step 18, the monitor will display the exercise values up to the current time, as well as the current menu, on screen 20, which is shown in FIG. 2.

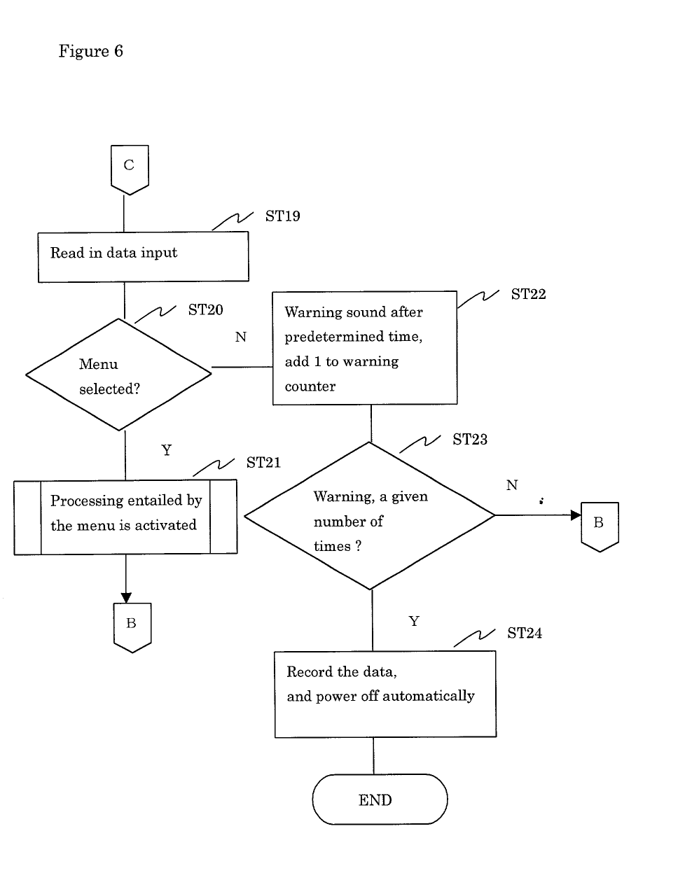

In FIG. 6, the monitor determines whether the user has, while he is resting, selected a new menu, or whether he has completely stopped exercising, and goes into its final processing. In Step 19, the monitor reads in data input via the keys of operating unit 30. If a new menu is selected in Step 20, the processing entailed by the menu is activated in Step 21, and we go back to Step 02, from which the processing continues. If a new menu is not selected in Step 20, in Steps 22 and 23 a warning is sounded a given number of times, the essential data are stored in a non-volatile memory and the power supply is disconnected. In Step 23, if the warning has not yet been output the specified number of times, we return to Step 02 and processing resumes.

With this processing flow, the amount of exercise performed in each session is computed and recorded (as exercise record data), and the exercise result data are accumulated in the exercise monitor. Although it is not shown in the flow chart, these exercise result data are displayed on screen 20, as was mentioned earlier. They may be transmitted by phone to the insurance company's data base after a specified period of time has elapsed, or they may be sent to a specified home page via the internet. In this case, since the person reporting the data is the person who stands to benefit from them, the insurance company would develop its own encoding scheme to safeguard the accuracy of the data. The aforesaid exercise result data would then appear on the screen in encoded form so that the person calling in the data would be unable to alter them. The insurance company decodes these encoded exercise result data and uses the exercise record to confer benefits such as a premium discount on its insured.

Processing to Verify that the Monitor is Being Worn

We shall now present, with reference to FIGS. 7 through 11, a detailed explanation of the processing to verify that the monitor is being worn, which constitutes SUB 200 in FIG. 4. By “processing to verify that the monitor is being worn” is meant processing to insure that the insured who stands to benefit is actually wearing the exercise monitor rather than fraudulently creating exercise record data by some dishonest means.

For the exercise monitor related to this invention, we disclose the following five techniques to verify that the monitor is actually being worn. The invention, of course, is not limited to these techniques, which are meant only to serve as examples.

(B1) Giving an instruction which only the person wearing the monitor can detect. For example, the exercise monitor could vibrate and instruct the wearer to perform a given exercise. For this the monitor need not be in contact with the person's skin.

(B2) Recognizing a characteristic signal which can be detected only when the person is exercising while wearing the monitor. Precise analysis of an acceleration signal detected by the monitor can yield a waveform which is definitive for an exercise performed while the person is wearing the monitor (e.g., the waveform produced when the person is walking). For this the monitor need not be in contact with the person's skin.

(B3) Verifying a physiological rhythm which is synchronized with a rhythm generated by the exercise monitor. This requires that the monitor detect a physiological signal and that it be in contact with the person's skin. We shall discuss a means to verify a synchronous rhythm shortly.

(B4) Verifying acceleration signals which are synchronized with a rhythm generated by the exercise monitor. We shall discuss a means to verify this synchronization shortly. This method does not require that the monitor be in contact with the person's skin.

(B5) Continuously detecting a physiological signal. This method requires that the monitor be in contact with the person's skin. We shall next explain, in order, the five aforesaid methods to verify that the monitor is being worn.

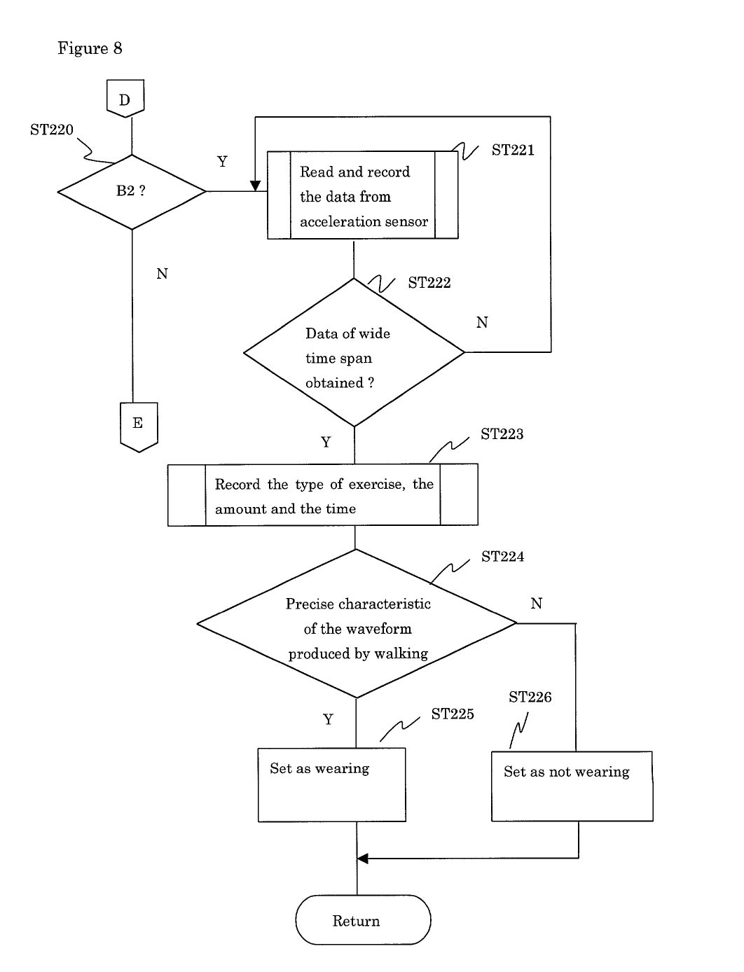

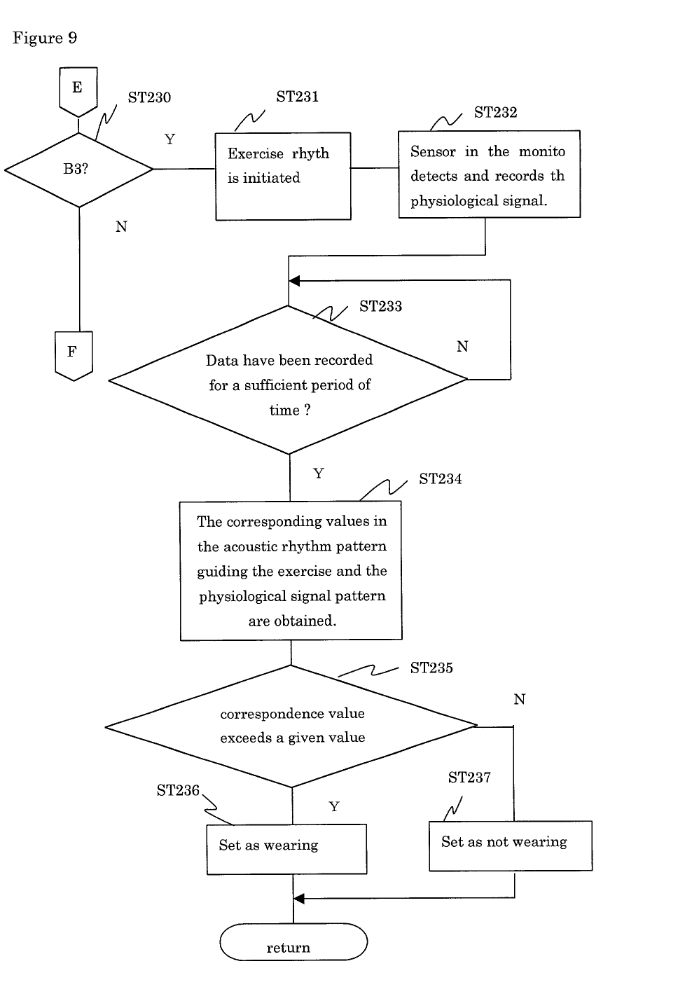

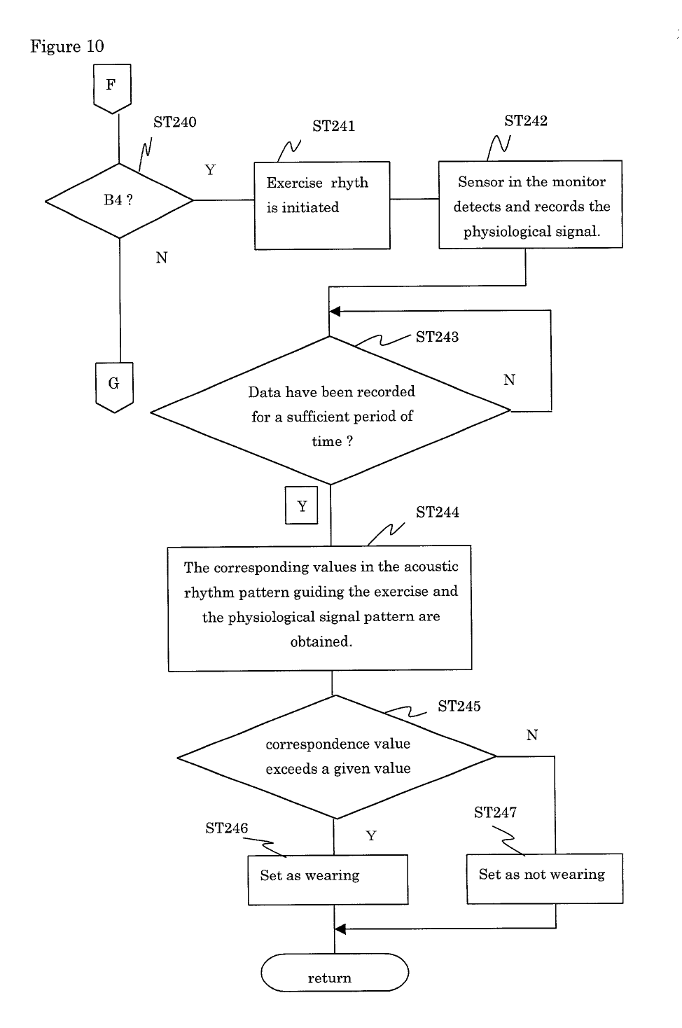

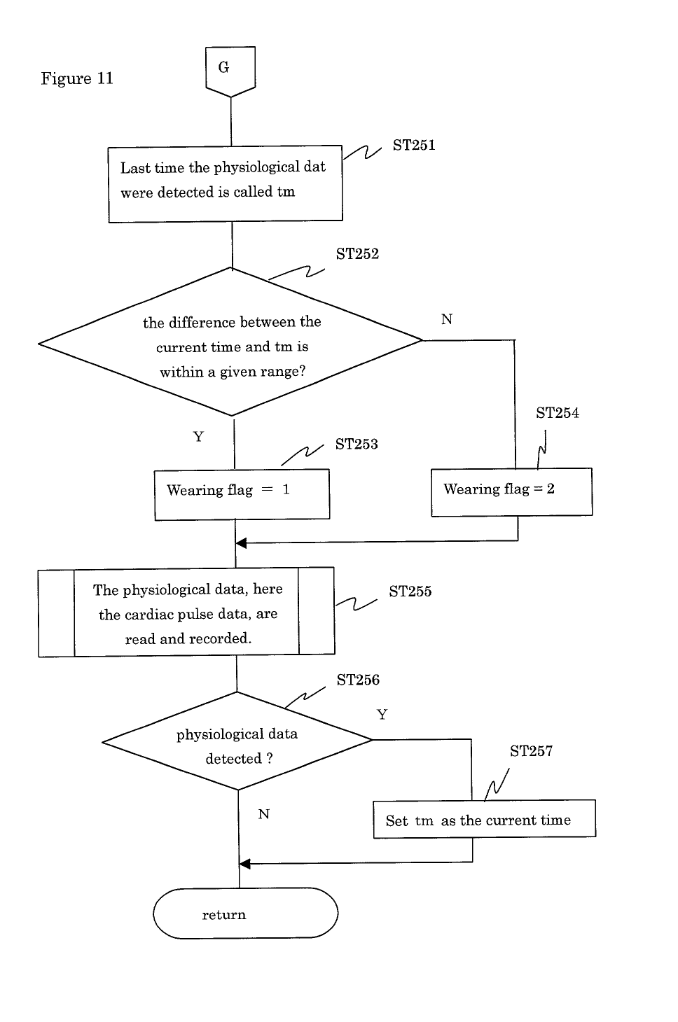

In FIG. 7, when SUB 200, the processing to verify that the monitor is being worn, begins, a number is read out in Step 201 to designate which method is to be used for the verification. This number will be one of B1 through B5 in the flow chart. It is set by the insurance company before the exercise monitor is distributed. It is not specifically necessary to set one method only; more than one method could be used for verification. Method B1 is shown in FIG. 7, B2 in FIG. 8, B3 in FIG. 9, B4 in FIG. 10 and B5 in FIG. 11.

Method B1

If Method B1 is recognized in Step 210 in FIG. 7, the exercise monitor outputs a vibration to indicate that the wearer should perform a specific exercise. When the wearer feels this vibration, he performs an exercise determined ahead of time. For example, if the monitor is a pedometer, he might take ten steps, pause five seconds, and take another ten steps. In Step 212, an acceleration sensor detects this exercise, and the data are read and recorded. If the person is not actually wearing the monitor, he will be unable to produce this sort of conditioned response; so the test can be used to verify that he is wearing the monitor. In Step 213, the monitor determines whether a specified period of time has elapsed. If it has, in Step 214 it records the type of exercise, the amount and the time. In Step 215, the monitor determines whether the exercise which the wearer was instructed to perform has been detected. In Steps 216 and 217, it establishes either that the exercise was detected or that it was not. The monitor then leaves the verification routine and proceeds to Step 05. The specified exercise may be determined beforehand, as described above, or a different request may be displayed on screen 20 each time verification is attempted.

Method B2

When Method B2 is recognized in Step 220 of FIG. 8, the processing will be the same as in Method B1 from Step 221 through Step 226, with the exception of Step 224. This method differs from B1 in regard to Step 224, where instead of detecting a specified number of steps registered on a pedometer, as described in B1, the monitor detects a more precise characteristic of the waveform produced by walking. Checking the waveform characteristic of walking enables a more rigorous verification.

Method B3

When Method B3 is recognized in Step 230 of FIG. 9, the monitor requests that the wearer perform some exercise which is synchronized with a specified rhythm that it generates. The monitor can then detect whether the person is wearing it by checking the rhythm of the physical data it records. In Step 231, the process of outputting an exercise rhythm is initiated. The person must exercise at the same rhythm. In Step 232, a sensor in the monitor detects and records the physiological signal. If data have been recorded for a sufficient period of time in Step 233, the corresponding values in the acoustic rhythm pattern guiding the exercise and the physiological signal pattern are obtained in Step 234. In Step 235, the monitor determines whether the correspondence value exceeds a given value. In Steps 236 and 237, if the correspondence exceeds the given value, the monitor concludes that the person is wearing it, and if it does not exceed the value, it concludes that the person is not wearing it. This concludes the verification processing. The “synchronized physiological signal rhythm” might, for example, be the person's heart rhythm. The determination made in the aforesaid Steps 234 and 235, as to whether the acoustic rhythm to guide the exercise can be correlated with the waveform which is detected, will be discussed in detail shortly, as will the details of the flow, which comprises SUB 400.

Method B4

When Method B4 is recognized in Step 240 of FIG. 10, the monitor determines whether or not the person is wearing it in a similar fashion to that of Method B3, in Steps 241 through 247. However, instead of the physiological sensor used in Method B3, this method uses an acceleration sensor. The exercise monitor bases its judgement on an acceleration signal which is synchronized with a rhythm it generates. It might, for example, base its determination on a speed of walking which is synchronized with a rhythm indicated by a pedometer. All other steps are identical to those of Method B3. Like Steps 234 and 235 in FIG. 9, the determination made in the aforesaid Steps 244 and 245, as to whether the acoustic rhythm to guide the exercise can be correlated with the waveform which is detected, will be discussed in detail shortly, as will the details of the flow, which comprises SUB 400.

Method B5

FIG. 11 illustrates the process of verifying whether the person is wearing the monitor using Method B5. This method entails continuously monitoring data such as the cardiac pulse. If the pulse is not detected for a period which exceeds a specified duration, the monitor concludes that the person has taken it off. It records in the memory the last time the cardiac pulse was detected and calculates the difference between that time and the next time the pulse is detected. If the result exceeds a given value, the monitor concludes that it has been removed. In Step 251, the last time the physiological data were detected is called tm. In Step 252, the monitor determines whether the difference between the current time and tm is within a given range. If it is, the monitor concludes that it has not been removed. In Step 253, the verification processing is completed, the monitor verifies that the person is still wearing it, and it sets the “monitor being worn” flag to “1”. If the time difference is outside the given range, the verification processing ends with Step 254, the monitor cannot verify that it is being worn, and it sets the “monitor being worn” flag to “2”. In Step 255 the physiological data, here the cardiac pulse data, are read and recorded. If the physiological data have been detected in Step 256, the time they were last detected is changed to the current time in Step 257. The verification processing is completed, and we proceed to Step 05 in FIG. 4. In Step 04 it was determined that the verification process had been completed. In Step 06 the person's identity is checked, and we go back to Step 13 in FIG. 5 to read and record the physiological data. The monitor repeatedly checks as described above to determine whether it has been taken off.

Verifying the Person's Identity

We shall next explain, with reference to FIGS. 12 and 13, the details of the processing to verify the person's identity which constitute SUB 300, shown in FIG. 4. “Verifying the person's identity” refers to determining whether the person who is wearing the exercise monitor is actually the insured who is the beneficiary or some other person. In relation to the exercise monitor according to this invention, two methods are given to verify identity; however, the invention should not be construed as limited to these methods only, which are meant merely as examples.

(A1) Analyzing an answer to a question. This method entails determining whether this is the correct person by whether his answers to questions such as requests for his PIN number, address or name are correct. This method does not require the detection of physiological data, so it does not require that a sensor terminal be attached to the person's skin. However, it does require a keyboard or some other means to input data.

(A2) Using physiological data which identify an individual. The monitor obtains a fingerprint, voiceprint, ECG waveform or cardiac pulse and verifies the person's identity by how well these data match a previously recorded set. For an ECG waveform or a cardiac pulse, a sensor must be attached to the person's skin.

In FIG. 12, when SUB 300, the processing to verify the person's identity, begins, in Step 301 the monitor reads out a number to designate which method will be used to verify identity. This number, which is either A1 or A2 in this flow chart, is set by the insurance company before the exercise monitor is shipped. There is, of course, no need specifically to set one of the methods. Both may be used to verify identity, or one of a number of methods may be selected randomly to prevent anyone from impersonating the insured and passing the verification dishonestly. Method A1 is shown in FIG. 12 and Method A2 in FIG. 13.

Method A1

In FIG. 12, when SUB 300, the subroutine to verify the person's identity, begins, a number is read out in Step 301 to designate which method will be used to verify the person's identity. This number, which is either A1 or A2 in this flow chart, is set by the insurance company before the exercise monitor is shipped. We have already explained that there is no specific need to set one of the methods, and that a number of methods may be used to verify identity. Method A1 is shown in FIG. 12 and Method A2 in FIG. 13.

In Step 302 of FIG. 12, when Method A1 is recognized, the exercise monitor displays a question such as “What is your PIN number?” on screen 20 and outputs a warning sound via speaker 40. The user enters his answer via the keyboard in Steps 303 and 305. The key input is read in Step 304 and compared with previously recorded reference data in Step 306. If in Step 307 the monitor determines that this is not the correct person, it sets a flag to indicate this in Step 309. If it determines that this is the correct person, it sets a flag to indicate this in Step 310. If in Step 305 a specified time elapses, a time out is declared, and a “verification incomplete” flag is set in Step 308. We then leave SUB 300 and proceed to Step 07.

Method A2

If Method A1 is not selected in Step 302 of FIG. 12, we move to Step 311 in FIG. 13 and verify the person's identity using Method A2. In Step 311 of Method A2, the screen displays information needed to collect physiological data to check the person's identity. At the same time, a warning sound is emitted. After the physiological data are entered in Step 312, they are read in Step 314. By “physiological data” are meant a fingerprint pattern, a cardiac pulse pattern, an ECG pulse pattern or the like. In Step 315 the monitor determines how well the recorded pattern fits a reference pattern. In Steps 316 and 317 flags representing the results of this determination are set. We then leave SUB 300 and proceed to Step 07.

Recording the Type, Amount and Time of Exercise

The acoustic pattern which guides the exercise is compared to the recorded physiological pattern described in Steps 234 and 235 of FIG. 9 or the signal pattern output by the acceleration sensor, which is described in Steps 244 and 245 of FIG. 10. The correlation between these two patterns is determined as follows.

The acoustic rhythm of the sounds to guide the exercise, which are repeated throughout period T, is called timing waveform f(t). The timing waveform of the physiological data obtained by the acceleration sensor or the physical measurement unit is called g(t). The correlation function Φ(t) is then obtained by the following formula.

$$Φ ( t ) = {\int }_{- T / 2}^{T / 2} f ( t + τ ) g ( τ ) ⅆ τ$$

Φ(t) not only represents the goodness of fit between timing waveform f(t) and the form of g(t), but is a function influenced by the amplitude as well; it must therefore be normalized. If we call the energy of f(t) f0 and the energy of g(t) g0, we obtain normalized results by the following formulas.

$$f0 = {\int }_{- T / 2}^{T / 2} f ( τ ) f ( τ ) ⅆ τ$$

$$g0 = {\int }_{- T / 2}^{T / 2} g ( τ ) g ( τ ) ⅆ τ$$

Φ(t) represents the normalized cross-correlation function of the normalized function. If we represent this as [symbol] (t), we get the following formula.

ψ(t)=Φ(t)/sqrt(f0×g0)

where sqrt (x) is the function which obtains the square root of x.

The maximum value of [symbol] (t) for the time interval [−T/2, T/2] is called [symbol] max.

We compare [symbol] max with the threshold value TH and determine whether it is over the threshold value.

[symbol] max is over TH: The two patterns are similar.

[symbol] max is under TH: The two patterns are not similar.

SUB 400 to Record the Type, Amount and Time of Exercise

Using the aforesaid logic as our premise, we shall now explain the actual processing flow in SUB 400 with reference to FIG. 14. In Step 401 the data record g(t) representing the output of the acceleration sensor is read out of the memory. In Step 402, the N types of fundamental waveform models fm1 (t), fm2 (t) . . . fmn(t) for the various types of exercise are read out. In Steps 404, 405 and 406, the logic discussed above is used to obtain the normalized cross-correlation function for g (t) and the kth fundamental waveform model fmk (t). The maximum value of this function we shall call Mk. We apply the same processing to each exercise model, obtaining M1 through Mn for the models. In Step 407, we determine the type of exercise from the number of the fundamental waveform model in M1 through Mn which has the greatest value. In Step 408, the maximum value of the cross-correlation function for the fundamental waveform model with the number obtained as described above and g(t) is recorded as the intensity of the exercise. Finally, in Step 409, the starting and ending points of the time interval recorded as g(t) are recorded so as to correspond with the type and amount of exercise. In this way we can obtain exercise data which have been compared to every type of exercise rhythm.

System to Manage Insurance Premiums

In this section we shall disclose a business system which employs the aforesaid exercise monitor. In such a system, when the insurance company or subcontracting health guidance company recognizes the fact that the insured has exercised regularly for a fixed period of time and so is making an effort to maintain his own health, they will discount his premium by a given amount.

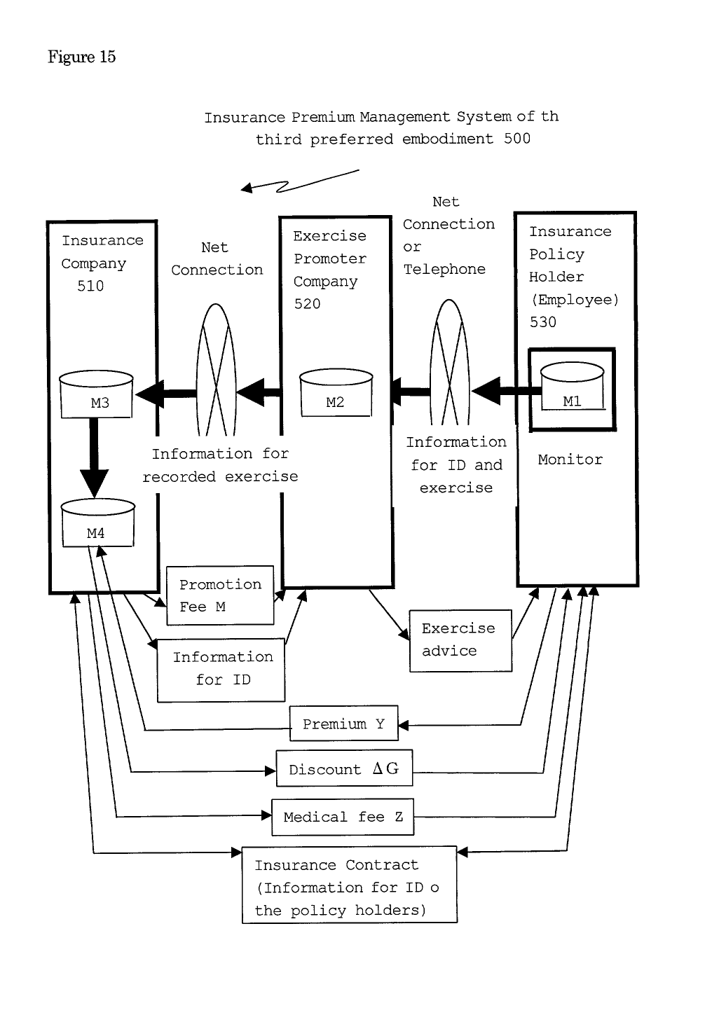

FIG. 15 shows premium management system 500, which is related to the third preferred embodiment of this invention. In this embodiment, when insurance company 510 and policyholder 530 enter into a contract, the insurance company stipulates that if the policyholder exercises sufficiently, his premium will be discounted. Insurance company 510 gives policyholder 530 an exercise monitor 1 or 2 according to this invention to keep track of how much he exercises. The policyholder gives the insurance company the telephone number of the location where he usually works out (for example, a fitness center) or the number of the cell phone he will carry when working out. Every time the policyholder exercises to maintain his fitness, he wears the exercise monitor. And each time he exercises, the type and amount of exercise will be stored in storage device M in his monitor.

Insurance company 510 or its agent, health guidance company 520, will telephone the policyholder and, via the internet, collect the exercise data recorded by his exercise monitor, which has verified his identity and the fact that he is actually wearing the monitor. As is shown in the drawing, policyholder 530 may himself phone in his data, for example by sending them to the file M2 for exercise records provided on the homepage of health guidance company 520; or he may report them orally by phone. As has been discussed, the exercise monitor according to this invention has a number of functions, including verifying the person's identity, verifying that he is actually wearing the monitor and encoding the data he is to report, which eliminate the possibility that false results will be reported.

Once health guidance company 520, to which insured 530 has sent his ID and exercise data, has finished inspecting the ID and exercise record, it sends them on via the internet to exercise record file M3 at the insurance company. Based on the data in file M3, insurance company 510 applies a discount of ΔG to the premium Y which was stored for the insured in premium data file M4 before he reported his exercise record, so that his future premium will be discounted by that amount. The medical expenses G of a policy-holder who continues exercising regularly for a period of time will with high probability decrease to G′. It is therefore possible to discount his premium Y by ΔG. To encourage insured 530 to use the exercise monitor, insurance company 510 can pay him an incentive bonus M. In this case, if the decrease in medical expenses (G−G′) is larger than (ΔG+M), the insurance company can actually use this system to increase its profit. Health guidance company 520 can evaluate whether insured 530 is exercising appropriately and instruct him in appropriate exercises. It will then receive a promotion fee M from the insurance company as its service fee. Insured 530 receives a premium discount of ΔG from the insurance company and is instructed in his exercise routine by the health guidance company so as to maintain his fitness.

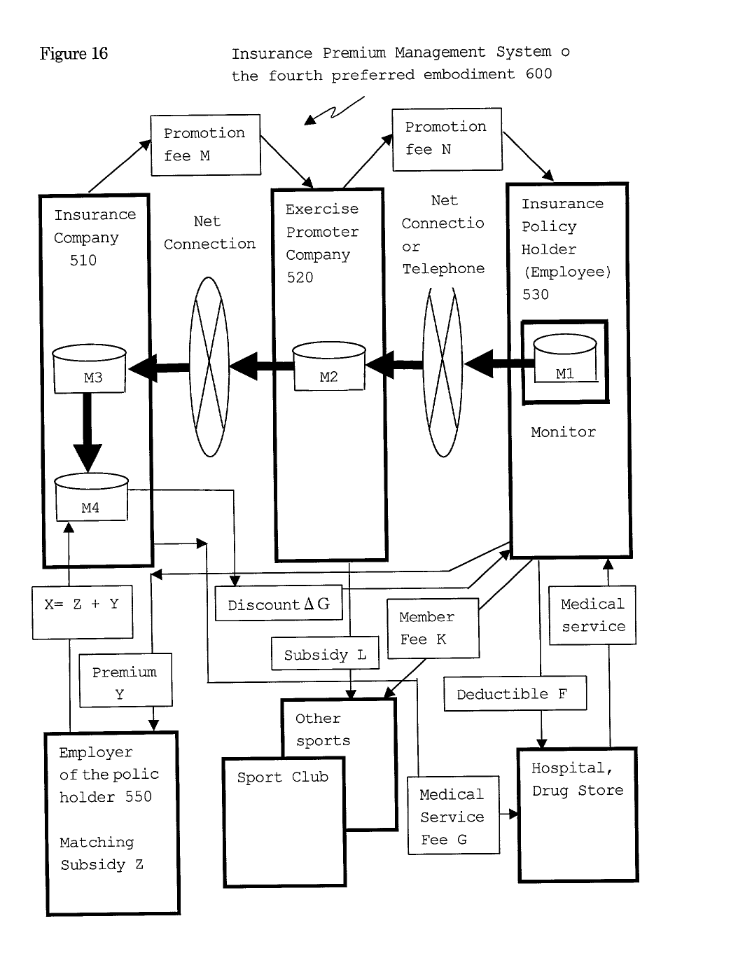

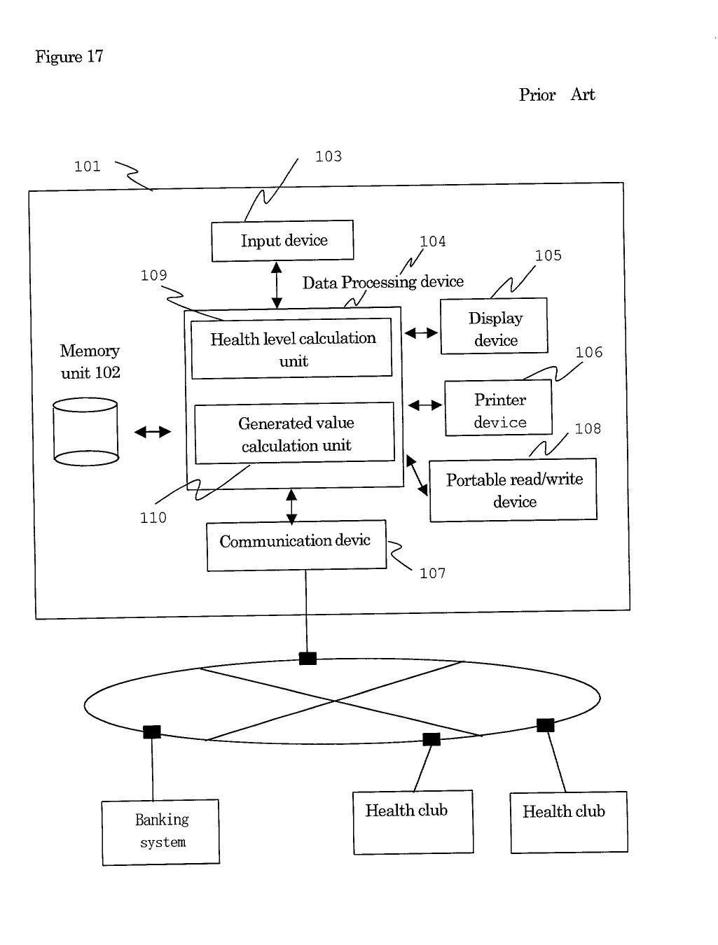

FIG. 16 shows insurance premium management system 600, which is related to the fourth preferred embodiment of this invention. This is a business model for the development of the third embodiment. Employer 550 pays the insurance company which covers the policyholder (i.e., his employee) a premium X which consists of the employee's share Y and the employer's share Z. Insurance company 510 pays medical expenses G to a hospital or pharmacy when the insured is ill. To reduce the amount it must pay as medical expenses G, insurance company 510 gives policyholder 530 an exercise monitor 1 or 2 according to this invention. When the insured exercises and becomes fit, his medical expenses G will decrease, and the insurance company will not have to pay as much. The insurance company can also pay a promotion fee M to health guidance company 520 to encourage insured 530 to exercise. In this case, we would expect that medical expenses G will be reduced by ΔG. If M is less than ΔG, the insurance company will make a profit.

Health promotion company 520 subsidizes a portion (L) of insured 530's health club membership fee in return for access to his exercise record data. As a result, the employee can belong to a health club at a lower out-of-pocket cost. We would predict that this would cause health club memberships to increase. Even if the insured does not join a health club but prefers to exercise on his own, he can still wear the exercise monitor, and the fact that he is performing sufficient exercise can be understood from the data collected by the monitor. IF exercise promotion company 520 is monitoring the insured's exercise regime, it will pay him a promotion fee N. In this case, if the total of company 520's management expenses, promotion fee N and membership fee subsidy L is less than promotion fee M, its income, it will turn a profit. Exercise promotion company 520 can increase its profit by selling the exercise monitor to the insured and by servicing used monitors.

Effects of the Invention

As was discussed above, this exercise monitor does not simply record exercise data as prior art monitors did. Its identity verification function not only certifies, by requiring the user to input a PIN number or the like, that this is the insured, but also determines with certainty that this is not someone impersonating the insured. This function prevents a person other than the insured from padding the exercise record data. Because the user's identity is checked immediately after he stops exercising, only exercise performed by the insured will be recorded. This device would allow interested parties to construct an effective system to manage health insurance premiums, in which the insurance company or a health guidance company which it subcontracts with would provide the insured with this exercise monitor to encourage him to exercise voluntarily. If the insured records a certain amount of exercise in a given period of time, the insurance company acknowledges that he is making an effort to keep fit, and it discounts his premium.

What is claimed is:

1. An exercise monitor, comprising:
a motion sensor adapted to measure an exercise motion of a user;
identity check means for verifying said user's identity, said identity check means operable in an active mode; and
recording means for recording an output data of said motion sensor;
wherein said recording means records said exercise motion as valid data only after said identity check means has verified said user's identity when said identity check means is operated in said active mode.

2. An exercise monitor according to claim 1, wherein said verifying said user's identity is processed without notice.

3. An exercise monitor according to claim 1, wherein said verifying said user's identity is processed by said user in said exercise motion right after a predetermined length of time has passed.

4. An exercise monitor according to claim 1, wherein said verifying said user's identity is processed by asking said user one or more specific questions and allowing said user to input one or more correct answers for said one or more specific questions.

5. An exercise monitor according to claim 4, wherein said one or more questions are selected out of a plurality of questions previously registered in said exercise monitor.

6. An exercise monitor according to claim 4, wherein said verifying said user's identity is processed by asking said user one or more specific questions without notice.

7. An exercise monitor according to claim 4, wherein said one or more specific questions are delivered by telephone or said questions are stored in said exercise monitor in advance.

8. An exercise monitor according to claim 4, wherein said one or more correct answers for said one or more specific questions are personal key words which only said user knows.

9. An exercise monitor according to claim 1, wherein said verifying said user's identity is processed by physiological data of said user.

10. An exercise monitor according to claim 9, wherein said physiological data is a fingerprint pattern or voiceprint pattern of said user.

11. An exercise monitor according to claim 1, further comprising, in addition to said identity check means, a wearing check means to verify that said monitor is actually being worn by said user;
wherein said recording means records said exercise motion as valid data only after said wearing check means has verified that said monitor is actually being worn by said user.

12. An exercise monitor according to claim 11, wherein said wearing check means is activated based on an instruction which said user can not obtain unless said user is actually wearing said exercise monitor adjacent to the skin of said user.

13. An exercise monitor according to claim 12, wherein said instruction which said user can not obtain unless said user is actually wearing said exercise monitor adjacent to the body of said user, is to vibrate a portion of the skin of said user.

14. An exercise monitor according to claim 11, wherein said wearing check means is processed based on a characteristic signal which can be detected only when said user is exercising while wearing said exercise monitor.

15. An exercise monitor according to claim 14, wherein said exercise monitor is provided with a step counter equipped with said motion sensor to detect a walking motion, and said motion sensor can verify said wearing by a motion characteristic of walking.

16. An exercise monitor according to claim 11, wherein said wearing check means is actually being worn by said user, is processed if there is a high correlation between physiological data of said user which is obtained only if said monitor is actually being worn by said user, and an output of said exercise monitor.

17. An exercise monitor according to claim 16, wherein said physiological data of said user is a synchronized exercise rhythm generated by said exercise monitor and obtained only if said monitor is actually being worn by said user, and said verifying is processed if there is a high correlation between said synchronized exercise rhythm and a pulse wave data of said user which is said output of said exercise monitor.

18. An exercise monitor according to claim 11, wherein said wearing check means is processed if there is a high correlation between acceleration data of said user which is obtained only if said monitor is actually being worn by said user, and an output of said motion sensor.

19. An exercise monitor according to claim 18, wherein said acceleration data of said user is a synchronized exercise rhythm generated by said exercise monitor and obtained only if said monitor is actually being worn by said user, and said verifying is processed if there is a high correlation between said synchronized exercise rhythm and an acceleration of said user's body which is related to said output of said motion sensor.

20. An insurance premium management system which provides a predetermined amount of benefits to an insured person who is recognized by an insurance company as an insured person who has achieved a predetermined amount of exercise, and thereby reduces medical expenses of said insured person, by using an exercise monitor to recognize that said insured person has achieved said predetermined amount of exercise, said exercise monitor; comprising:
a motion sensor adapted to measure an exercise motion of a user;
identity check means for verifying said user's identity, said identity check means operable in an active mode; and
recording means for recording an output data of said motion sensor;
wherein said recording means records said exercise motion as a valid data only after said identity check means has verified said user's identity when said identity check means is operated in said active mode.

21. An insurance premium management system according to claim 20, wherein said predetermined amount of benefits includes a premium discount.

22. An insurance premium management system according to claim 20, wherein said exercise monitor further comprises, in addition to said identity check means, a wearing check means to verify that said monitor is actually being worn by said user, and said recording means records said exercise motion as a valid data only after said wearing check means has verified that said monitor is actually being worn by said user.

23. An insurance premium management system according to claim 21, wherein said premium discount is lower than an actual reduction of said medical expenses for said insurance company by usage of said exercise monitor.

24. An insurance premium management system according to claim 22, wherein data of an achieved amount of exercise is provided to said insurance company by a health guidance company, and said health guidance company generates an information value, said health guidance company is paid for within a saving value range for said insurance company, said saving value range is defined by a difference between an amount of premium discounts and an actual reduction of said medical expenses.

25. An insurance premium management system according to claim 22, wherein said health guidance company further provides a healthcare guidance for said insured person.
